(19) United States
(12) Patent Application Publication
(10) Pub. No.: US 2005/0080252 A1
(43) Pub. Date: Apr. 14, 2005

(54) Process for preparing antiviral nucleoside derivatives

(75) Inventors: Andrew John Briggs, Mountain View, CA (US); Charles Alois Dvorak, Palo Alto, CA (US); Anthony Prince, (US); Keith McCarthy, Old Lyme, CT (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 10/939,819

(22) Filed: Sep. 13, 2004

Related U.S. Application Data
(60) Provisional application No. 60/502,074, filed on Sep. 11, 2003.

(51) Int. Cl. C07H 19/056

(52) U.S. Cl. 536/28.7

(57) ABSTRACT

Process and novel intermediates for preparing 2-aminocarboxylic acid esters of the 5-hydroxymethyl group of levovirin (1-(3S,4R-dihydroxy-5S-hydroxymethyl-tetrahydro-furan-2S-yl)-1H-[1,2,4]triazole-3-carboxylic acid amide; Id: R¹═R²═R³═H) and acid addition salts thereof. The present process provides the monoesters selectively in high purity with increased efficiency with reduced number of production steps. The process involves condensation of a cyclopentylidene levovirin compound with a N-urethane-N-carboxylic anhydride and subsequent deprotection to directly provide the hydrochloride salt of the product. The mono esters are useful for treatment of viral diseases and are absorbed more efficiently than the parent compound.

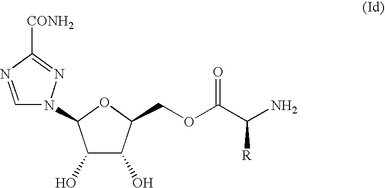

### CROSS REFERENCE TO PRIOR APPLICATION

This application claims benefit under Title 35 U.S.C. 119(e) of U.S. Provisional Application No. 60/502,074, filed Sep. 11, 2003, which is hereby incorporated by reference in its entirety. The related application filed herewith, Non-Nucleoside Reverse Transcriptase Inhibitors (J. P. Dunn et al.), is hereby incorporated by reference in its entirety.

### FIELD OF THE INVENTION

The invention relates to a novel process to prepare prodrugs of levovirin, or an acid addition salts, solvate or hydrate thereof. Levovirin is useful for treating Hepatitis C Virus (HCV) mediated diseases. More specifically the invention relates to a process for preparing hydrochloride salts of 3,4-dihydroxy-5-(3-methyl-[1,2,4]triazol-1-yl)-tetrahydro-furan-2-ylmethyl 2-amino-carboxylates. The invention further relates to novel chemical intermediates useful in the above process and to a process for preparing the intermediates.

### BACKGROUND OF THE INVENTION

Hepatitis C virus (HCV) is responsible for a large proportion of the chronic liver disease worldwide and accounts for 70% of cases of chronic hepatitis in industrialized countries. The global proportion of hepatitis C is estimated to average 3% (ranging from 0.1% to 5.0%); there are an estimated 170 million chronic carriers throughout the world. There is a continuing need for effective therapeutic agents against HCV. Standard therapy for hepatitis C infection presently consists of combination therapy with an antiviral, ribavirin, and an immunomodulatory interferon derivative.

WO 01/45509 (J. Lau et al.) discloses L-nucleosides with in vivo antiviral activity against HCV. Levovirin (1-(3S,4R-dihydroxy-5S-hydroxymethyl-tetrahydro-furan-2S-yl)-1H-[1,2,4]triazole-3 carboxylic acid amide; Ia:R¹═R²═R³═H), is the L-isomer of the antiviral nucleoside ribavirin (Ib). Unlike ribavirin, levovirin does not have direct detectable antiviral activity; however, levovirin stimulates immune responses by enhancing antiviral Th1 cytokine expression. Levovirin appears to lack toxicity associated with ribavirin.

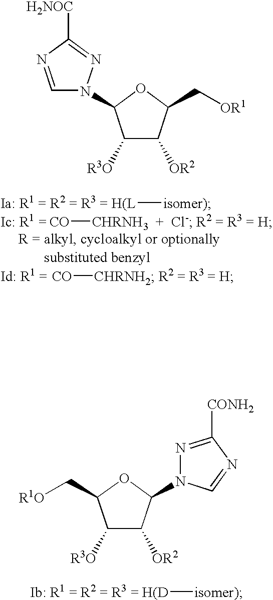

While nucleoside derivatives frequently possess high levels of biological activity, their clinical utility is often hampered by suboptimal physical properties and limited bioavailability requiring large doses at frequent intervals to maintain therapeutically effective levels. Chemical modification of the nucleoside can alter the physicochemical properties of the compound and improve the efficiency and selectivity of drug delivery.

Colla et al. (J. Med. Chem. 1983 26:602-04) disclose the preparation of water soluble ester derivatives of acyclovir by convention coupling with a diimide and base. L. M. Beauchamp et al. (Antiviral Chem & Chemother. 1992 3(3):157-64) disclose eighteen amino acid esters of the antiherpetic drug acyclovir and their efficiencies as prodrugs of acyclovir including: the glycyl, D,L-alanyl, L-alariyl, L-2-aminobutyrate, D,L-valyl, L-valyl, DL-isoleucyl, L-isoleucyl, L-methionyl, and L-prolyl ester. According to the authors the L-valyl ester of acyclovir was the best prodrug of the esters investigated. These esters were prepared by methods similar to those employed by Colla et al.

EP 0 375 329 (L. M. Beauchamp) disclosed the preparation of the bis-isoleucine ester of gangciclovir by contacting an optionally protected amino acid or a functional equivalent thereof with a coupling agent such as DCC optionally in the presence of catalytic base. The product so obtained contained about 90% of the diester and about 10% of the monoester.

U.S. Pat. No. 6,215,017 B1 (C. A. Dvorak et al.), U.S. Pat. No. 6,218,568 B1 (C. A. Dvorak et al.) and U.S. Pat. No. 6,040,446 (C. A. Dvorak et al.) disclose processes and novel intermediates useful for the preparing the mono-L-valine ester of 2-(2-amino-1,6-dihydro-6-oxo-purin-9-yl)methoxy-1,3-propanediol (ganciclovir). WO 94/29311 (W. P. Jackson) discloses a process for esterification acyclovir and ganciclovir derivatives with 2-oxa-4-aza-cycloalkane-1,3-dione compounds (N-carboxyanhydrides, NCA).

WO 00/23454 (A. K. Ganguly et aL) disclose bioreversible prodrugs of ribavirin Ib. Compounds in which the 5-hydroxy of Ib is esterified to natural and unnatural amino acids are disclosed. Amino acid esters were prepared by SP 435 lipase catalyzed reaction of O-acyl acetone oxime esters of amino acids. U.S. Pat. No. 6,423,695 (R. Tam et al.) disclose methods of treating a patient with a virus infection by administering amidine prodrugs of ribavirin

WO 01/68034 (G. Wang et al.) disclose bioreversible phosphorylated and non-phosphorylated prodrugs of levovirin. 5-Acyl and 2,3,5-triacyl compounds are disclosed and 5-amino acid esters are also described generically. U.S. Ser. No. 60/432,108 discloses acylated prodrugs of levovirin.

### SUMMARY OF THE INVENTION

The present invention provides a process to prepare 5-acyloxynucleoside compounds. The individual steps which comprise specific embodiments of the present invention are depicted in the reaction sequence in Scheme I. The present invention further provides an efficient process for the isolation of acid addition salts of the acyloxy compounds wherein R is methyl, ethyl, n-propyl, iso-propyl, n-butyl, iso-butyl, sec-butyl, C₃₋₇ cycloalkyl or phenyl optionally substituted with a substituent selected from the group consisting of C₁₋₃ alkyl, C₁₋₃ alkoxy and halogen. In scheme 1, the acylation step is depicted with an N-carboxyanhydride; however the present process includes other activated N-protected amino acids sufficient reactive to esterify an alcohol. R^{1a} and R^{1b} are individually are alcohol protecting groups or R^{1a} and R^{1b} together are a vic-diol protecting group and R⁴ is hydrogen or an N-protecting group. The full scope of R^{1a}, R^{1b} and R⁴ is more fully disclosed in the detailed description of the Process. Steps. 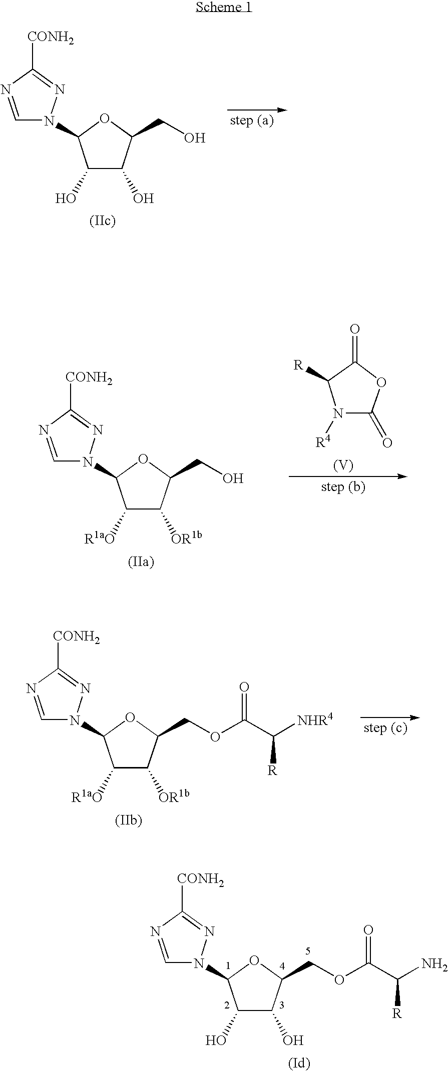
step (a) cyclopentanone, trimethylorthoformate, p-TsOH; step (b) cat TEA, THF; step (c) HCI, H₂O, toluene, isopropanol

### DETAILED DESCRIPTION OF THE INVENTION

The individual steps which comprise specific embodiments of the present invention are depicted by the reaction sequence in Scheme I wherein R is methyl, ethyl, n-propyl, iso-propyl, n-butyl, iso-butyl, sec-butyl, C₃₋₇ cycloalkyl or phenyl optionally substituted with a substituent selected from the group consisting of C₁₋₃ alkyl, C₁₋₃ alkoxy and halogen. R^{1a} and R^{1b} taken together are R³R²C where R³ and R² are (CH₂)₄₋₆, or taken independently are lower alkyl or R² is optionally substituted phenyl or lower alkoxy and R³ is hydrogen. R^{1a} and R^{1b} taken independently are trialkylsilyl or an aralkyl radical. One skilled in the art will realize that a large number of hydroxyl protecting groups have been identified which could be used without departing from the spirit of the invention and any protecting group which can be removed under conditions which allow for the efficient isolation of an acid addition salt are within the scope of the invention. R⁴ is an amino protecting group or hydrogen. A large number of amino-protecting groups are known which can be used interchangeably. Urethanes represent one group of amine protecting groups which are useful in the present process and commonly used urethane protecting groups include the tert-butoxy carbonyl and benzyloxycarbonyl radicals. When an N-carboxyanhydride is used as the acylating agent no amine protecting group is required.

In one embodiment of the present invention there is provided a process for preparing a compound according to formula Id comprising the steps of (i) contacting IIa with an acylating agent to afford IIb; and, (ii) contacting IIb with a deprotecting reagent to afford Id, an acid addition salt, a solvate or a hydrate thereof wherein R, R^{1a}, R^{1b} and R⁴ are as defined hereinabove.

In another embodiment of the present invention there is provided a process for preparing a compound according to formula Id comprising the steps of (i) contacting IIa, wherein R^{1a} and R^{1b} are together are R²CR³ and R² and R³ together are C₃₋₆ alkylene, independently are lower alkyl, or R² is phenyl and R³ is hydrogen, with an acylating agent to afford IIb; and, (ii) contacting IIb with a deprotecting reagent to afford Id, an acid addition salt, a solvate or a hydrate thereof, wherein R and R⁴ are as defined hereinabove.

In another embodiment of the present invention there is provided a process for preparing a compound according to formula Id comprising the steps of (i) contacting IIa, wherein R^{1a} and R^{1b} together are R²CR³ and R² and R³ together are C₃₋₆ alkylene, with an acylating agent; and, (ii) contacting IIb with a deprotecting reagent to afford Id, an acid addition salt, a solvate or a hydrate thereof wherein R and R⁴ are as defined hereinabove.

In another embodiment of the present invention there is provided a process for preparing a 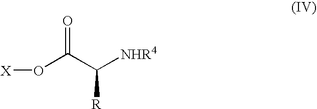
compound according to formula Id comprising the steps of (i) contacting IIa with an activated N-protected alpha amino acid according to formula IV wherein X is an activating group rendering the acid sufficiently reactive to esterify an alcohol, R⁴ is an N-urethane protecting group to afford IIb; and, (ii) contacting IIb with a deprotecting reagent to afford Id, an acid addition salt, a solvate or a hydrate thereof, and wherein R, R^{1a} and R^{1b} are as defined hereinabove.

In another embodiment of the present invention there is provided a process for preparing a comnpound according to formula Id comprising the steps of (i) contacting IIa with a compound of formula IV wherein X is an activating group sufficiently reactive to esterify an alcohol, R is iso-propyl and R⁴ is boc or cbz; and, (ii) contacting IIb with a deprotecting reagent to afford Id, an acid addition salt, a solvate or a hydrate thereof, and wherein R^{1a} and R^{1b} are as defined hereinabove.

In another embodiment of the present invention there is provided a process for preparing a 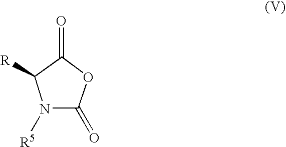
compound according to formula Id comprising the steps of (i) contacting IIa with a N-carboxyanhydride (NCA) according to formula V wherein R⁵ boc or cbz to afford IIb; and, (ii) contacting IIb with a deprotecting reagent to afford Id, or an acid addition salt, a solvate or a hydrate thereof and wherein R, R^{1a} and R^{1b} are as defined hereinabove.

In another embodiment of the present invention there is provided a process for preparing a compound according to formula Id comprising the steps of (i) contacting IIa with a compound of formula V wherein R is iso-propyl and R⁵ is boc to afford IIb; and, (ii) contacting IIb with a deprotecting reagent to afford Id, an acid addition salt, a solvate or a hydrate thereof, and wherein R^{1a} and R^{1b} are as defined hereinabove.

In another embodiment of the present invention there is provided a process for preparing a compound according to formula Id comprising the steps of (i) contacting IIa, wherein R^{1a} and R^{1b} together are R²CR³ and R² and R³ together are C₃₋₆ alkylene; with an acylating agent to afford IIb; and, (ii) deprotecting IIb with a mixture of toluene, isopropanol and aqueous hydrochloric acid to afford the hydrochloric acid addition salt of Id, or a solvate or hydrate thereof, wherein R is as defined hereinabove.

In another embodiment of the present invention there is provided a process for preparing a compound according to formula Id comprising the steps of (i) contacting IIa, wherein R^{1a}and R^{1b} together are R²CR³ and R² and R³ together are (CH₂)₄, with an NCA according to formula V wherein R is iso-propyl and R⁵ is boc to afford IIb; and, (ii) contacting IIb with a mixture of toluene, isopropanol and aqueous hydrochloric acid to afford the hydrochloric acid addition salt of Id, or a solvate or hydrate thereof.

In another embodiment of the present invention there is provided a process for preparing a compound according to formula Id comprising the steps of (i) contacting 1-((2S,3S,4R,5S)-3,4-dihydroxy-5-hydroxymethyl-tetrahydro-furan-2-yl)-1H-[1,2,4]triazole-3-carboxylic acid amide (IIc) with a vic-diol protecting group to afford a compound of formula IIa; (ii) contacting IIa with an acylating agent to afford IIb; and, (iii) contacting IIb with a deprotecting reagent to afford Id, an acid addition salt, a solvate or a hydrate thereof wherein R, R^{1a}, R^{1b}, and R⁴ are as defined hereinabove.

In another embodiment of the present invention there is provided a process for preparing a compound according to formula Id comprising the steps of contacting (i) 1-((2S,3S,4R,5S)-3,4-dihydroxy-5-hydroxymethyl-tetrahydro-furan-2-yl)-1H-[1,2,4]triazole-3-carboxylic acid amide (IIc) with a vic-diol protecting group R²C(═O)R³ wherein R² and R³ together are C₃₋₆ alkylene, independently are lower alkyl or R² is phenyl or alkoxy and R³ is hydrogen to afford a compound of formula IIa; (ii) contacting IIa with an acylating agent to afford IIb; and, (iii) contacting IIb with a deprotecting reagent to afford Id, an acid addition salt, a solvate or a hydrate thereof, wherein R, and R⁴ are as defined hereinabove.

In another embodiment of the present invention there is provided a process for preparing a compound according to formula Id comprising the steps of contacting (i) 1-((2S,3S,4R,5S)-3,4-dihydroxy-5-hydroxymethyl-tetrahydro-furan-2-yl)-1H-[1,2,4]triazole-3-carboxylic acid amide (IIc) with a vic-diol protecting group is R²C(═O)R³ wherein R² and R³ is C₃₋₆ alkylene to afford a compound of formula IIa wherein R^{1a} and R^{1b} together are R²CR³ and R² and R³ together are C₃₋₆ alkylene; (ii) contacting IIa with an acylating agent to afford IIb; and, (iii) contacting IIb with a deprotecting reagent to afford Id, or an acid addition salt, or a solvate or hydrate thereof wherein R and R⁴ are as defined hereinabove.

In another embodiment of the present invention there is provided a process for preparing a compound according to formula Id comprising the steps of contacting (i) 1-((2S,3S,4R,5S)-3,4-dihydroxy-5-hydroxymethyl-tetrahydro-furan-2-yl)-1H-[1,2,4]triazole-3-carboxylic acid amide (IIc) with a vic-diol protecting group is R²C(═O)R³ wherein R² and R³ is C4−5 alkylene to afford a compound of formula IIa wherein R^{1a} and R^{1b} together are R²CR³ and R² and R³ together are C₄₋₅ alkylene; (ii) contacting IIa with a N-carboxyanhydride according to formula V wherein R is iso-propyl, R⁵ boc or cbz; and, (ii) contacting IIb with a mixture of toluene, isopropanol and aqueous hydrochloric acid to afford the hydrochloric acid addition salt of Id, or a solvate or hydrate thereof.

In another embodiment of the present invention there is provided compounds formula XI 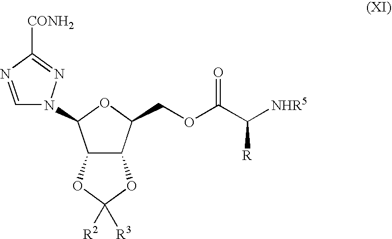
wherein R is methyl, ethyl, n-propyl, iso-propyl, n-butyl, iso-butyl, sec-butyl, C₃₋₇ cycloalkyl or phenyl optionally substituted with a substituent selected from the group consisting of C₁₋₃ alkyl, C₁₋₃ alkoxy and halogen; R² and R³ together are (CH₂)ₙ, or R² is alkoxy and R³ is hydrogen; R⁵ is hydrogen, boc or cbz; and, n is 1 to 3 which are useful intermediates in the synthesis of compounds of formula Id.

One skilled in the art will recognize that although the stereochemistry of the amino acid was depicted in the natural S-configuration in scheme 1 and formulae IV and V, the process could be carried out with unnatural R-configuration in the same manner and the claims encompass a process in which the amino acid, or the amino acid derivative, possesses either configuration.

### Definitions

Unless otherwise stated, the following terms used in this Application, including the specification and claims, have the definitions given below. The phrase “a” or “an” entity as used herein refers to one or more of that entity; for example, a compound refers to one or more compounds or at least one compound. As such, the terms “a” (or “an”), “one or more”, and “at least one” can be used interchangeably herein.

In general, the systematic nomenclature used in this Application is based on AUTONOM™ v.4.0, a Beilstein Institute computerized system for the generation of IUPAC systematic nomenclature.

The phrase “as defined hereinabove” refers to the broadest definition provided in the Summary of the Invention.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “arylalkyl” or “aralkyl” as used herein denotes the radical R′R″—, wherein R′ is an aryl radical as defined herein, and R″ is an alkylene radical as defined herein with the understanding that the attachment point of the arylalkyl moiety will be on the alkylene radical. Examples of arylalkyl radicals include, but are not limited to, benzyl, phenylethyl and 3-phenylpropyl.

The term “alkoxy group” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “C₁₋₁₀ alkoxy” as used herein refers to an-O-alkyl wherein alkyl is C₁₋₁₀.

The term “alkylene” as used herein denotes a divalent linear or branched saturated hydrocarbon radical, having from four to six carbons inclusive, unless otherwise indicated. Examples of alkylene radicals include propylene, butylene, pentylene or hexylene.

The term “cycloalkyl” as used herein denotes a saturated carbocyclic ring containing 3 to 7 carbon atoms, i.e. cyclopropyl, cyclobutyl, cyclopentyl, cyclohexyl or cycloheptyl. “C₃₋₇ cycloalkyl” as used herein refers to an cycloalkyl composed of 3 to 7 carbons in the carbocyclic ring.

The term “alkanol” as used herein means an HO-alkyl group, wherein alkyl is as defined above such as methanol, ethanol, n-propanol, i-propanol, n-butanol, i-butanol, t-butanol, including their isomers.

The term “urethane” as used herein refers to a group ROC(═O)NH—where the nitrogen atom is an alpha-amino group of an amino acid. R in the urethane is alkyl as used herein preferably tert-butyl (boc) or R is benzyl (cbz). An equivalent definition for “urethane” as used herein is an alkoxycarbonyl or benzyloxycarbonyl linked to an amino group.

The term “orthoester” as used herein refers to a group RC(OR′)3 wherein R is alkyl or hydrogen and R′ is alkyl.

The term “aprotic ( or nonpolar) solvent” means organic solvents such as diethyl ether, ligroin, pentane, hexane, cyclohexane, heptane, octane, benzene, toluene, dioxane, tetrahydrofuran, carbon tetrachloride.

The term “derivative” of a compound as used herein means a compound obtainable from the original compound by a simple chemical process.

The term “acylating agent” as used herein refers to either an anhydride, acid halide or an activated derivative of an N-protected alpha amino acid. The term “anhydride” as used herein refers to compounds of the general structure RC(O)—O—C(O)R wherein R is an N-protected alpha amino. The term “acid halide” as used herein refers to compounds of the general structure RC(O)X wherein X is a halogen. The term “activated derivative” is as defined below.

The term “activated derivative” of a compound as used herein refers to a transient reactive form of the original compound which renders the compound active in a desired chemical reaction, in which the original compound is only moderately reactive or non-reactive. Activation is achieved by formation of a derivative or a chemical grouping within the molecule with a higher free energy content than that of the original compound, which renders the activated form more susceptible to react with another reagent. In the context of the present invention activation of the carboxy group is of particular importance and corresponding activating agents or groupings which activate the carboxy group are described in more detail below. Of particular interest for the present invention is an amino acid anhydride which is an activated form of an amino acid which renders the amino acid (especially L-valine) susceptible to esterification. An example of an activated derivative of L-valihe is the compound of V wherein R is iso-propyl and R⁴ is Boc.

The term “protecting group” as used herein refers to a chemical group that (a) preserves a reactive group from participating in an undesirable chemical reaction; and (b) can be easily removed after protection of the reactive group is no longer required. For example, the benzyl group is a protecting group for a primary hydroxyl function.

The term “amino-protecting group” as used herein refers to a protecting group that preserves a reactive amino group that otherwise would be modified by certain chemical reactions. The definition includes the formyl group or lower alkanoyl groups with 2 to 4 carbon atoms, in particular the acetyl or propionyl group, the trityl or substituted trityl groups such as the monomethoxy-trityl group, dimethoxytrityl groups such as the 4,4′-dimethoxytrityl, the trichloroacetyl group, the trifluoroacetyl group, the silyl group, the phthalyl group, and N-urethanes. Preferred amino-protecting groups are N-urethanes such as the N-benzyloxycarbonyl group (cbz) derived from benzylchlorocarbonate or N-alkoxycarbonyl group, e.g. tert-butoxycarbonyl which is prepared by reaction with di(t-butyl)dicarbonate.

The term “hydroxyl protecting group” or “alcohol protecting group” means a protecting group that preserves a hydroxy group that otherwise would be modified by certain chemical reactions. In the context of the present invention, a “vic-diol” protecting group refers to a moiety which simultaneously protects two hydroxyls on adjacent carbon atoms. A hydroxy-protecting group can be an ether, an ester-, or silane that can be removed easily after completion of all other reaction steps, such as a lower acyl group (e.g., the acetyl or propionyl group or a dimethyl-t-butylsilyl group), or an aralkyl group (e.g., the benzyl group, optionally substituted at the phenyl ring). A “vic-diol protecting group” is usually an aldehyde or ketone, e.g. acetone, benzaldehyde, or cyclopentanone, which facilely and reversibly forms a dioxolane. A cyclic orthoester formed by contacting an acyclic ortho ester with a vic-diol to form a 2-alkoxy-dioxolane also is an effective protecting group within the scope of the present invention.

The term “deprotecting reagent” as used herein refers to reagents contacted with the levovirin derivative to remove the amino- and vic-diol protecting groups. Reagents and protocols for deprotection are well known and can be found in Greene and Wuts or in Harrison and Harrison (infra). One skilled in the chemical arts will appreciate that on occasion protocols must be optimized for a particular molecule and such optimization is well with the ability of one skilled in these arts.

The term “optional” or “optionally” as used herein means that the subsequently described event or circumstance may but need not occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

As used herein, the term “treating”, “contacting” or “reacting” when referring to a chemical reaction means to add or mix two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “nucleoside” as used herein refers to a nitrogenous heterocyclic base linked to a pentose sugar by a glycosidic bond at C-1. Naturally occurring bases include uracil, thymine, cytosine, adenine and guanine and naturally occurring sugars are ribose and 2-deoxyribose. The term nucleoside further encompasses compounds in which the sugar and/or the nitrogenous base have been chemically modified.

Although specific methods for producing 5′-acyloxy levovirin derivatives are described below, numerous modifications and alternative process steps will be apparent to those skilled in the art. Accordingly, this description and these examples are to be construed as illustrative only and is for the purpose of teaching those skilled in the art novel processes for producing 5′-acyloxy levovirin derivatives. These processes may be varied substantially without departing from the spirit of the invention and the exclusive use of all modifications which come within the scope of the appended claim is reserved.

### Abbreviations

Boc tert-butoxycarbonyl
Bn benzyl
cbz benzyloxycarbonyl
DCE 1,2-dichloroethane
DEAD diethyl azodicarboxylate
DLAD di-iso-propylazodicarboxylate
DMF N,N-dimethylformamide
EtOAc ethyl acetate
Et₂O diethyl ether
EtOH ethanol
MeCN acetonitrile
MeOH methanol
NCA N-carboxyanhydride
NMP N-methylpyrrolidone
psi pounds per square inch
pyr pyridine
rt room temperature
TEA triethylamine
TFA trifluoroacetic acid
THF tetrahydrofuran
TLC thin layer chromatography
TsOH p-toluenesulfonic acid monohydrate
UNCA N-urethane-N-carboxyanhydride
vic vicinal

### Process Steps

### Step (a) Hydroxyl Protection

To insure selective esterification of the 5-hydroxy of levovirin (IIc) the 2′-and 3′-secondary hydroxy groups of the ribosyl moiety must be protected. Protecting groups for vicinal diols often convert the diol into a dioxolane or dioxane ring. Most commonly these protecting groups include aldehydes and ketones which readily form dioxolanes. Ketones which have found particular utility as diol protecting groups include acetone and C₅₋₇ cycldalkanones. Reverson of a ketal to the diol is accomplished with aqueous acid and an organic cosolvent. Benzaldehyde readily forms acetals with Vic diols which can be deprotected by hydrogenolysis or acidic hydrolysis. Methoxy substitution on the benzaldehyde increases the rate of acidic hydrolysis and also permits cleavage of the dioxolane under oxidative conditions, e.g. Ce(NH₄)₂(NO₃)₆. Nitrobenzaldehydes afford dioxolanes which can be photochemically cleaved. Cyclic orthoesters, e.g. ethoxymethylene acetal have been utilized as diol protecting groups. These compounds can be cleaved under mild acidic conditions; however the initial product is an ester which must be hydrolyzed to regenerate the diol. The cyclic analog 2-oxacyclopentylidene ortho ester affords the diol directly upon acid hydrolysis. Cyclic carbonates and cyclic boronates also have found some utility as diol protecting groups. Any of these diol protecting groups could be adapted to the present process. More detailed information regarding protection and deprotection of alcohols can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 John Wiley and Sons, 1971-1996. General reviews of the preparation of nucleoside compounds providing discussions of strategies for nucleoside synthesis also have been published (A M Michelson The Chemistry of Nucleosides and Nucleotides Academic Press, New York 1963; L Goodman Basic Principles in Nucleic Acid Chemistry Ed. P O P Ts'O, Academic Press, New York 1974, Vol. 1, chapter 2; Synthetic Procedures in Nucleic acid Chemistry Ed W W Zorbach and R S Tipson, Wiley, New York, 1973, Vol. 1 and 2; H. Vorbruggen and C. Run-Polenz Handbook of Nucleoside Chemistry Wiley, New York. 2001). The above references are incorporated herein by reference in their entirety.

Preferably, the protecting group is selected to allow the facile isolation of a acid addition salt of the amino substituted alkanoyl ester with minimal additional purification. The selection of a protecting group will also be influenced by the need to avoid rigorous deprotection conditions which could lead to partial hydrolysis of the ester, epimerization or exchange of the acyl group with newly deprotected hydroxy groups.

### Step (b) Esterification

Prior to carrying out the esterification step, the amino group of the amino acid must be protected to prevent undesirable amide formation. Numerous N-protecting groups have been developed which can be selectively cleaved under a variety of conditions. Protection strategies for coupling amino acids have been extensively reviewed (see e.g., M. Bodanszky, Principles of Peptide Synthesis, Springer Verlag, New York 1993; P. Lloyd-Williams and F. Albericio Chemical Methodsfor the Synthesis of Peptides and Proteins CRC Press, Boca Raton, Fla. 1997). These references are incorporated herein in their entirety. The various amino-protecting groups useful in this invention include N-benzyloxy-carbonyl- (cbz), tert-butoxy-carbonyl (Boc), N-formyl- and N-urethane-N-carboxy anhydrides which are all commercially available (SNPE Inc., Princeton, N.J., Aldrich Chemical Co., Milwaukee, Wis., and Sigma Chemical Co., St. Louis, Mo.) N-urethane amino-protected cyclic amino acid anhydrides are also described in the literature (William D. Fuller et al., J. Am. Chem. Soc. 1990 112:7414-7416) which is incorporated herein by reference. While many of these could be effectively employed in the present process, preferred urethane protecting groups include the tert-butoxycarbonyl or the benzyloxycarbonyl.

The amino acid must also be activated prior to carrying out the esterification step. Protocols for efficient coupling of N-protected amino acids have been refined and extensively optimized (M. Bodanszky supra; P. Lloyd-Williams and F. Albericio supra). At least 1 equivalent of the protected amino acid and 1 equivalent of a suitable coupling agent or dehydrating agent, e.g., 1,3-dicyclohexylcarbodiimide or salts of such diumides with basic groups, N-ethyl-N′-(3-(dimethylamino) propyl)carbodiimide hydrochloride, should be employed from the start. Other dehydrating agents such as N,N′-carbonyldiimidazole, trifluoroacetic anhydride, mixed anhydrides, acid chlorides may be used. Numerous additives have been identified which improve the coupling efficiency and limit racemization of the alpha-amino acid including, 1-hydroxybenzotriazole and 3-hydroxy-3,4-dihydro-4-oxo-1,2,3-benzotriazine (W. König and R. Geiger Chem. Ber.1970 788:2024 and 2034), N-hydroxysuccinimide (E. Wunsch and F. Drees, Chem. Ber. 1966 99:110), 1-hydroxy-7-azabenzotriazole (L. A. Carpino J. Am. Chem. Soc. 1993 115:4397-4398). Aminium/uronium- and phosphonium HOBt/HOAt—based coupling reagents have been developed, e.g based peptide coupling reagents, e.g., 1-benzotriazol-1-yloxy-bis(pyrrolidino)uronium hexafluorophosphate (J. Xu and S. Chen Tetrahedron Lett. 1992 33:647), 1-benzotriazol-1-yloxy-N,N-dimethylmethananiminium hexachloroantimonate (P. Li and J. Xu, Tetrahedron Lett. 1999 40:3606), O-(7-azabenzotriazol-1-yl)-1,1,1,3,3-tetramethylammoniumuronium hexafluorophosphate (L. A. Carpino, J. Am. Chem. Soc.1993 115:4397), O-(7-azabenzotriazol-1-yl)-1,1,3,3-bis-(tetramethylene)uronium hexafluorophosphate (A. Erlich et al. Tetrahedron Lett. 1993 34:4781), 2-(3,4-dihydro-4-oxo-1,2,3-benzotriazin-3-yl)-1,1,3,3-tetramethyluronium tetrafluoroborate (R. Knorr et al. Tetrahedron Lett. 1989 30:1927), 7-azobenzotriazolyoxy-tris-(pyrrolidino) hexafluorophosphate (F. Albericio et al., Tetrahedron Lett. 1997 38:4853), 1-benzotriazolyloxy-tris-(dimethylamino)phosphonium hexafluorophosphate (B. Castro et al. Tetrahedron Lett. 1976 14:1219) and, I-benzotriazoloxy-tris-pyrrolidinophosphonium hexafluorophosphate (J. Coste et al. Tetrahedron Lett. 1990 31:205).

Particularly useful for the present invention are N-urethane- N-carboxy anhydrides (UNCA's) (William D. Fuller et al. J. Am. Chem. Soc. 1990 112:7414-7416, which is incorporated herein by reference). Other protected amino acid N-carboxy anhydrides are described in PCT Patent Application WO 94/29311. UNCA's V do not require an activation step prior to coupling. The formation of CO₂ during the coupling irreversibly drives the coupling reaction to VI. However, one skilled in the chemical arts will recognize that a plurality of reagents can used to esterify the remaining 5-hydroxy group of IIa so long as the reaction proceeds selectively, in good yield without racemization of asymmetric centers. Alternative coupling reagents can be readily identified without undo experimentation.

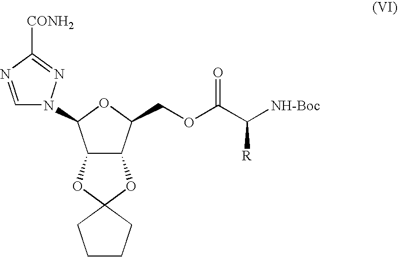

### Step (c) Deprotection Step

The N-amino acid protecting group and the ribosyl hydroxyl protecting group(s) are removed by de-protection reactions. The optimal conditions for removal of the protecting groups will depend on the particular protecting groups employed in the process. De-protection under acidic conditions ensures that the amino group liberated in the de-protection reaction will be protonated; i.e., the acid addition salt will be formed from at least stoichiometric amount of acid present. Isolating the compound of Formula (Id) as an acid addition salt helps to suppress racemization of the aminomethylene carbon and facilitate isolation of the product. Therefore, those examples given below show the de-protection step with the concomitant formation of an acid addition salt. The process can further comprise conversion of the acid addition salt to the free base or interchange with other pharmaceutically acceptable acid addition salts.

If the tert-butyloxycarbonyl group is being used as amino-protecting group, its removal is effected with acid such as aqueous HCl and an organic co-solvent or with trifluoroacetic acid neat. The former conditions will afford the hydrochloride salt directly while the latter conditions will afford the trifluoroacetate salt. The cyclopentylidene vic-diol protecting group can be removed simultaneously. The completion of the reaction can be monitored using conventional TLC analysis. The purification of the product and the isolation of a crystalline ester is carried out by recrystallization or other purification techniques such as liquid chromatographic techniques

If the cbz group is used as the amino-protecting group, its removal is effected by hydrogenolysis. The de-protection reaction is carried out by dissolving the product of the esterification step (c) in an inert solvent, preferably in an acidic solvent, using a hydrogenation catalyst such as palladium on carbon, or palladium hydroxide on carbon (Pearlman's catalyst), using elevated hydrogen pressure between 1 and 2000 psi (0.1-140 atm), preferably 20 to 200 psi (1.4-14 atm).

### Preparation of Salts

One of ordinary skill in the art will also recognize that the compound of formula Id may be prepared either as an acid addition salt or as the corresponding free base. If prepared as an acid addition salt, the compound can be converted to the free base by treatment with a suitable base such as ammonium hydroxide solution, sodium hydroxide, potassium hydroxide or the like. However, it is important to point out that the free base of formula Id is often more difficult to characterize than its acid addition salts.

Salts of acidic and basic compounds also can improve the physical properties of a parent compound. Ideally an active compound (i) possesses adequate chemical stability during the manufacturing process, (ii) is efficiently prepared, purified and recovered, (ii) exhibits acceptable solubility in pharmaceutically acceptable solvents, (iii) is amenable to handling (e.g. flowability and particle size) and formulation with negligible decomposition or change of the physical and chemical characteristics of the compound, (iv) exhibits acceptable long term chemical stability in the formulation. Salts wherein a low molar percent of the active ingredient is attributable to the counterion are highly desirable since they minimize the quantity of material which must be formulated and administered to provide a therapeutically effective dose. The pharmaceutical chemist, however, must identify these salt-forming agents, empirically since there is no reliable method to predict the influence of a salt species on the behavior of a parent compound in dosage formns. Effective screening techniques, which could simplify the selection process, are unfortunately lacking (G. W. Radebaugh and L. J. Ravin Preformulation. In, Remington: The Science and Practice of Pharmacy; A. R. Gennaro Ed.; Mack Publishing Co. Easton, Pa., 1995; pp 1456-1457).

The free base can be converted to another salt if required. When converting the free base to an acid addition salt, the compound is reacted with a suitable organic or inorganic acid. In the an acid addition salt-forming step, the free base is dissolved in a polar solvent such as water or a lower alkanol (preferably isopropanol) or mixtures thereof, and the acid is added in the required amount in water or in lower alkanol. Typically the free base is treated with an at least stoichiometric amount of an appropriate acid. The reaction temperature is usually kept at about 0° C. to 50° C., preferably at about room temperature. The corresponding salt precipitates spontaneously or can be brought out of the solution by the addition of a less polar solvent such as ether or hexane, removal of the solvent by evaporation or under vacuum, or by cooling the solution.

In present process treatment of IIb wherein R¹ is a ketal protecting group and R⁴ is boc with dilute hydrochloric acid provides the hydrochloride salt directly. The present process provides an improved method for the production of alpha-aminoacyl derivatives of levovirin (Id) which provides distinct advantages over other procedures. The desired compound Id is obtained as a crystalline product directly from the reaction mixture while residual by products remain in solution. The crystalline hydrochloride salt may be a solvate or hydrate. Furthermore the water-soluble salt is not hygroscopic and bulk density of the salt >0.4 gm/cm³ and large particle side allow for rapid filtration, drying and subsequent handling and processing. The formation of a pure crystalline product eliminates extra tedious purification steps from the process.

The foregoing discussion of the invention has been presented for purposes of illustration and description to enable those skilled in the art to more clearly understand and to practice the present invention. Although the description of the invention has included description of one or more embodiments and certain variations and modifications, other variations and modifications are within the scope of the invention, e.g., as may be within the skill and knowledge of those in the art, after understanding the present disclosure. The discussion should not be considered as limiting the scope of the invention, but merely as being illustrative and representative thereof. —The claims are intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges. or steps are disclosed herein, and without intending to publicly dedicate any patentable subject matter.

### Synthetic Reaction Parameters

Unless specified to the contrary, the reactions described herein take place at atmospheric pressure within a temperature range from 5° C. to 170° C. (preferably from 10° C. to 50° C.; most preferably at “room” or “ambient” temperature, i.e., about 20° C. to 30° C.) However, there are clearly some reactions where the temperature range used in the chemical reaction will be above or below these temperature ranges. Further, unless otherwise specified, the reaction times and conditions are intended to be approximate, e.g., taking place at about atmospheric pressure within a temperature range of about 5° C. to about 100° C. (preferably from about 10° C. to about 50° C.; most preferably about 20° C.-30° C.) over a period of about 1 to about 100 hours (preferably about 5 to 60 hours). Parameters given in the Examples are intended to be approximate. Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used without departing from the invention.

### EXAMPLE 1

### (S)-2-Amino-3-methyl-butyric acid (2S,3R,4S,5S)-5-(3-carbamoyl-[1,2,4]triazol-1-yl)-3,4-dihydroxy-tetrahydro-furan-2-ylmethyl ester; hydrochloride (via bocprotection)

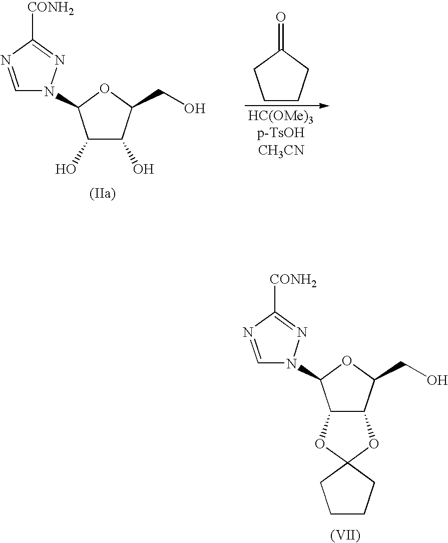 Step 1

A mixture of the levovirin (100 g; 0.453 mol), cyclopentanone (70 g; 0.839 mol), trimethylorthoformate (90 g; 0.857 mol) and pTsOH (6.8 g; 35.8 mmol) and MeCN (1.0 L) above reagents was heated to 35° C. with stirring. After two hours, temperature was increased to 40° C. for an additional two hours. The mixture became homogeneous and the reaction is considered complete at this point. The reaction mixture was made basic with 2.5 g triethylamine and the MeCN was removed in vacuo. The residue was stirred at 60°-65° C. and partitioned between 350 mL toluene, 120 mL MeOH and 600 mL water. The aqueous layer was separated and briefly distilled to remove methanol. On cooling the aqueous solution the cyclopentylidene crystallized and was filtered and dried to yield VII (97 g; 74.9% theory).
Step2

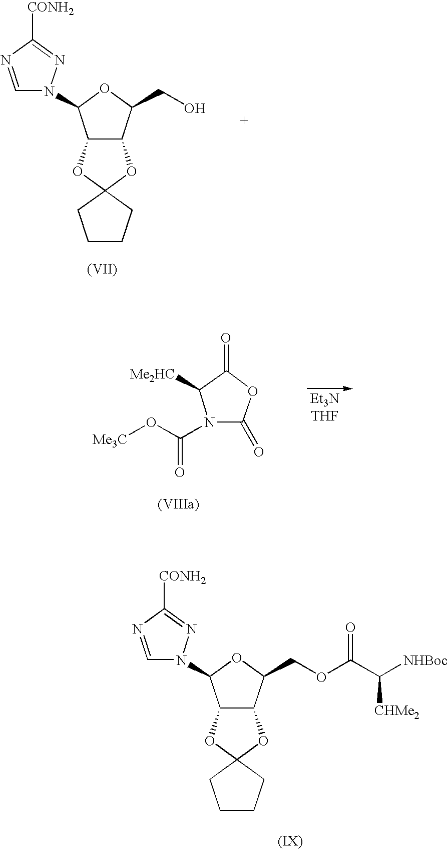

A solution of the 4-isopropyl-2,5-dioxo-oxazolidine-3-carboxylic acid tert-butyl ester (VIIIa; 450 g) in 1.25 L THF was slowly added to a mixture of the levovirin cyclopentylidene (VII; 500 g, 1.72 mol), TEA (18 g; 0.178 mol) and 3.75 L THF. The mixture was stirred for 16-24 h. The solution was concentrated in vacuo volatile solvents and the residue was dissolved in the EtOAc (2.5 L) and 55 mL of water was added to the solution. The mixture was poured onto a CELITE(G filter aid and saturated aqueous Na₂CO₃ solution (166 mL) was added to the stirred mixture. Toluene (3.5 L) was added after a brief period and the mixture was filtered. The filtrate was washed with two 650 mL portions of water. The organic phase was separated and filtered through the sodium carbonate and the filtrate is stripped to remove solvent, yielding 800 g of IX (98% theory) as viscous liquid which crystallized upon cooling.
Step3

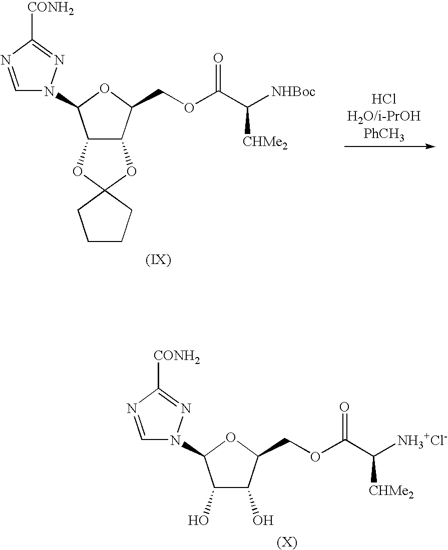

To a stirred solution of the levovirin cyclopentylidene (S)-2-tert-butoxycarbonylamino-3-methyl-butyrate (IX; 800 g; 1.57 mol) was dissolved in a mixture of the toluene (2.4 L) and isopropanol (500 mL) was added hydrochloric acid (315 g; 37%) diluted to a volume of 600 mL with water. The reaction mixture was stirred for 16-24 hours. The lower aqueous layer was separated, warmed to 35-50° C., and slowly diluted with isopropanol warmed to the same temperature (4.5 L). The mixture was cooled and stirred for several hours. The crystalline precipitate was collected by filtration and dried to yield levovirin valinate hydrochloride (X; 510 g).

### EXAMPLE 2

### S)-2-Amino-3-methyl-butyric acid (2S,3R,4S,5S)-5-(3-carbamoyl-[1,2,4]triazol-1-yl)-3,4-dihydroxy-tetrahydro-furan-2-ylmethyl ester; hydrochloride (Alternative coupling procedure)

A solution of the 4-isopropyl-2,5-dioxo-oxazolidine-3-carboxylic acid tert-butyl ester (VIIIa; 450 g) DMF (100 mL) DMF and toluene 2.0 L) was slowly added to a solution of the VII and TEA (26 g) in DMF (900 mL). After stirring several hours, the solution was slowly diluted with water (1.0 L). The organic layer was separated and diluted with toluene (500 mL). The organic phase was washed with H₂O (2×500 mL) and filtered through sodium carbonate (500 g). The organic layer is filtered over the sodium carbonate and the filtrate was concentrated in vacuo to yield 800 g of IX as viscous oil which crystallized upon cooling.

### EXAMPLE 3

### (S)-2-Amino-3-methyl-butyric acid (2S,3R,4S,5 S)-5-(3-carbamoyl-[1,2,4]triazol-1-yl)-3,4-dihydroxy-tetrahydro-furan-2-ylmethyl ester; hydrochloride (via cbz protection)

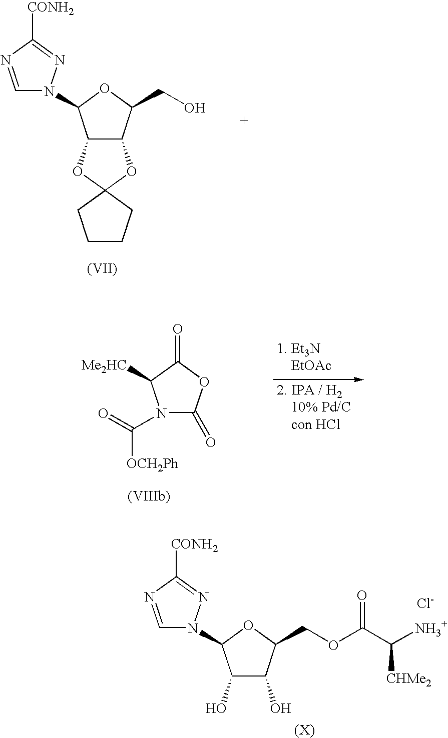 Step 1

A 500 mL 3-flask equipped with a mechanical stirrer, thermocouple and nitrogen inlet was charged with levovirin cyclopentylidene (VII; 30 g; 96.68 mmol) and 4-isopropyl-2,5-dioxo-oxazolidine-3-carboxylic acid benzyl ester (VIIIb) and EtOAc (240 mL). To the resulting slurry was added TEA (1.34 mL; 9.67 mmol). After 1.5 h the reaction mixture was a homogenous solution and the mixture allowed to stir overnight at rt. Approximately 170 mL of EtOAc was removed by vacuum distillation and replaced with a similar volume of isopropanol. The distillation was continued until another 170 mL of solvent was removed and another 200 mL of isopropanol was added.

Step 2

To the resulting solution was added 30 mL of H₂O, 10% Pd/C (1.32 g) and concentrated HCl (16.1 mL). The flask was fitted with an H₂-filled-balloon and maintained under H₂ atmosphere. The flask was periodically purged with a gentle vacuum to degas CO₂ and refilled with hydrogen. After 2.5 h another 30 mL of water was added to maintain a homogenous solution and periodic purging of CO₂ was continued. The reaction mixture was allowed to stir overnight. The following morning an additional 25 mL aliquot of water was added to dissolve the product and the catalyst was removed by filtering through CELITE®. Approximately 350 mL of isopropanol and water were removed by distillation and an additional 340 mL of IPA was added. The reaction mixture was allowed to cool slowly and seeded with a crystal of product at ca. 56° C. and cooling continued to rt and finally to 0° C. in an ice bath. The solid product was filtered and washed with IPA (75 mL). The solid was filtered and dried in a vacuum oven at 40° C. overnight to yield X (26.48 g; 72% theory).

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A process for preparing a compound according to formula Id wherein R is methyl, ethyl, n-propyl, iso-propyl, n-butyl, iso-butyl, sec-butyl, C₃₋₇ cycloalkyl or phenyl optionally substituted with a substituent selected from the group consisting of C₁₋₃ alkyl, C₁₋₃ alkoxy and halogen, and acid addition salts thereof, 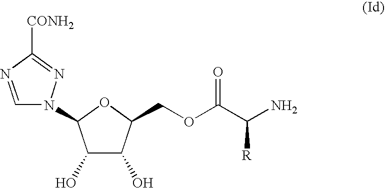
comprising the steps of
(i) contacting IIa with an acylating agent to produce IIb wherein R^{1a} and R^{1b} are a vic-diol or alcohol protecting groups and R⁴ is an amine protecting group; 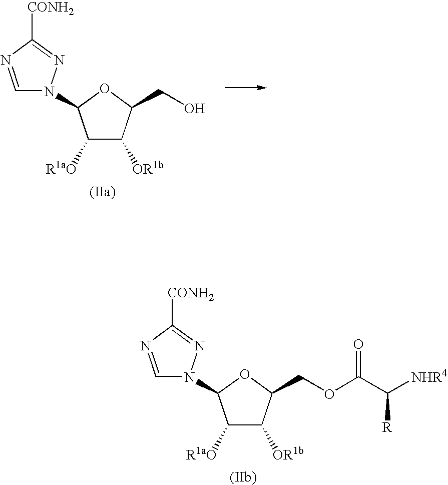
(ii) contacting IIb with a deprotecting reagent to afford Id or an acid addition salt or a solvate or hydrate thereof.

2. A process according to claim 1 wherein said vic-diol protecting group is R²CR³ wherein R² and R³ together are C₃₋₆ alkylene, independently are lower alkyl, or R² is phenyl or alkoxy and R³ is hydrogen.

3. A process according to claim 2 wherein said vic-diol protecting group is R²CR³ and R² and R³: together are (CH₂)ₙ and n is 3 to 6.

4. A process according to claim 1 where said acylating agent is an activated N-protected alpha amino acid according to formula IV wherein R is methyl, ethyl, iso-propyl, iso-butyl, sec-butyl, C₃₋₇ cycloalkyl or phenyl optionally substituted with a substituent selected from the group consisting of C₁₋₃ alkyl, C₁₋₃ alkoxy and halogen, X is an activating group suitable to esterify an alcohol, and R⁴ is N-urethane protecting group.

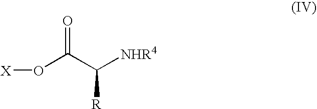

5. A process according to claim 4 wherein R is an iso-propyl group and R⁴ is boc or cbz.

6. A process according to claim 1 where said acylating agent is a N-carboxyanhydride according to formula V wherein R is as described above, R⁵ Boc or cbz.

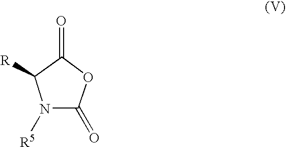

7. A process according to claim 6 where R is iso-propyl and R⁵ is Boc.

8. A process according to claim 1 wherein R^{1a} and R^{1b} together are R²CR³ and R² and R³ together are (CH₂)ₙ and n is 4 to 6, said deprotecting reagent comprises a mixture of toluene, isopropanol and aqueous hydrochloric acid and the hydrochloride salt of Id can be isolated from the reaction mixture.

9. A process according to claim 1 wherein said vic-diol protecting group is R²CR³ wherein R² and R³ together are (CH₂)₄, said acylating agent is a N-carboxyanhydride according to formula V wherein R is iso-propyl, R⁵ is Boc, said deprotecting reagent is a mixture of aqueous hydrochloric acid and toluene and said acid addition salt is a hydrochloride salt.

10. A process according to claim 1 further comprising the step of contacting 1-((2S,3S,4R,5S)-3,4-dihydroxy-5-hydroxymethyl-tetrahydro-furan-2-yl)-1H-[1,2,4]triazole-3-carboxylic acid amide (IIc) with a vic-diol protecting group to provide a compound of formula IIa;

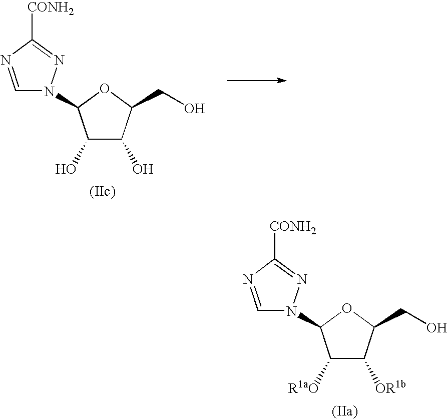

11. A process according to claim 10 wherein said vic-diol protecting group is R²C(═O)R³ wherein R² and R³ (i) together are C₄₋₆ alkylene, (ii) independently are lower alkyl or R² is phenyl or alkoxy and R³ is hydrogen or an orthoester derivative.

12. A process according to claim 11 wherein said vic-diol protecting reagent is R²C(═O)R³ and R² and R³ together are (CH₂)ₙ and n is 4-6.

13. A process according to claim 10 wherein said vic-diol protecting group is R²CR³ wherein R² and R³ together are (CH₂)₄, said acylating agent is a N-carboxyanhydride according to formula V wherein R is iso-propyl, R⁵ is Boc, deprotecting reagent comprises a mixture of toluene, isopropanol and aqueous hydrochloric acid and said acid addition salt is a hydrochloride salt.

14. A compound according to formula XI 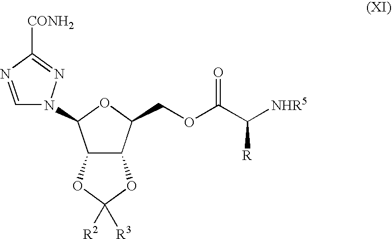
wherein
R is methyl, ethyl, n-propyl, iso-propyl, n-butyl, iso-butyl, sec-butyl, C₃₋₇ cycloalkyl or phenyl optionally substituted with a substituent selected from the group consisting of C₁₋₃ alkyl, C₁₋₃ alkoxy and halogen;
R² and R³ together are (CH₂)ₙ, or R² is alkoxy and R³ is hydrogen;
R⁵ is hydrogen, boc or cbz; and,
n is 1 to 3.
